(19) United States
(12) Patent Application Publication
(10) Pub. No.: US 2009/0259168 A1
(43) Pub. Date: Oct. 15, 2009

(54) Bandage

(76) Inventor: Eran Izhak Prizant, Hod Hasharon (IL)

Correspondence Address: Avia Kafri, 22 Shderot Chen St, Tel Aviv, 64166, IL

(21) Appl. No.: 12/083,497

(22) PCT Filed: Sep. 25, 2006
(86) PCT No.: PCT/IL06/01119

§ 371 Date: Apr. 11, 2008

(30) Foreign Application Priority Data
Oct. 16, 2005 (IL) 171456

(51) Int. Cl. A61H 1/00; A61F 13/02

(52) U.S. Cl. 604/22

(57) ABSTRACT

Sticky bandage (SB) for a topical treatment providing for massaging while applying cosmetic agents or drugs to a skin of a user is provided. The SB consists of a vibrating member and a layered SB base. The SB is electrically powered either by an internal battery or from an external source. The proximal layer (that is closer to the skin) of the SB base includes the active substances and is optionally replaceable. The vibrating member consists of piezoelectric ceramics, arrays of electromagnets, or miniature electric motor and programmable activation logic. Optionally the battery or the connector providing for electrical powering and an activation switch are installed in a separate housing that is electrically connected to the SB. An electrical safety circuit is provided to prevent an accidental activation of the vibrating member.

### FIELD OF THE INVENTION

The present invention relates in general to massaging devices and in particular to devices for skin care, therapeutic and/or cosmetic treatments.

### BACKGROUND OF THE INVENTION

Cosmetic agents that treat effects of aging, such as wrinkles and sagging of skin and underlying muscles, typically take the form of creams. Sticky bandages containing cosmetic agents or drugs for topical treatment are common in the marketplace. Massage is typically applied to stimulate blood circulation and enhance absorption of active ingredients of medications and/or cosmetic agents into the inner skin. Massage also effects a sensation of relief and warmth and contributes an enjoyable aspect to the cosmetic and/or therapeutic treatments associated with the administration of such ingredients.

Massage by itself is a known technique for treating skin. US patent application 20050187497A1 discloses an electrical impulse apparatus for facial massage. The disclosed apparatus consists of microprocessor controlling an impulse generator to generate pulsed signals to two electrodes on a pad. The pad is attached to the facial skin. Switch buttons provide for activation of the pulse generation and verification of the strength of the pulses. Electro-muscle stimulation can be applied to facial muscles, by means of the disclosed device, and thereby the tonus of facial muscles and the blood circulation in the surrounding area may be increased.

### BRIEF DESCRIPTION OF THE DRAWINGS

FIG. 1 is a schematic presentation of a facially applied sticky bandage (SB) according to the present invention;

FIG. 2 is an exploded view of the vibrating member of the SB shown in FIG. 1;

FIG. 3 is a sectional view an SB according to a preferred embodiment of the present invention;

FIG. 4 is a sectional view of an SB according to another preferred embodiment of the present invention.

### DETAILED DESCRIPTION OF THE PRESENT INVENTION

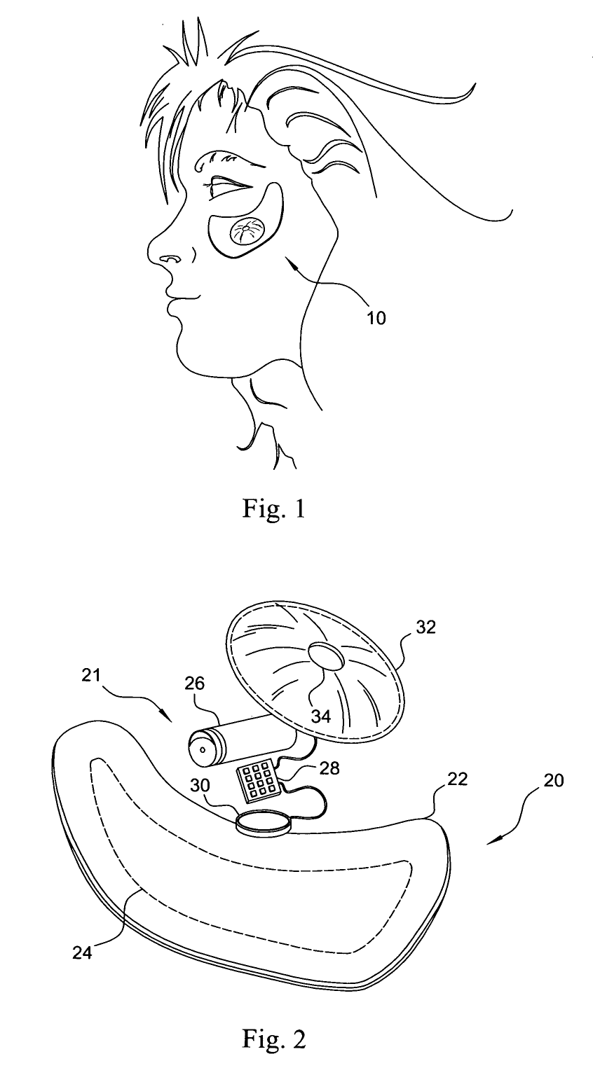

Reference is made to FIG. 1 schematically showing a sticky bandage (SB) of the present invention, applied to a facial skin of a user. SB 10, containing a drug or a cosmetic agent, is attached to facial skin for topical treatment. The SB has a proximal side, pointing towards the skin, and a distal side.

Reference is now made to FIG. 2 showing an expanded view of a vibrating member installed in the SB shown in FIG. 1. SB 20 consists of vibrating member 21 and SB base 22 containing one or more active substances. The active substance, such as a skin moisturizer, an anti-aging, or an anti-wrinkle, cream, is stored in a depression, the boundaries of which are designated by dashed line 24. Optionally SB base 22 consists of two layers that are removably attached to each other. The proximal layer of SB base 22 is replaceable and provides for storing the active substance or substances. Vibrating member 21 is firmly attached to, or embedded in, the distal layer of SB base 22 as is further described infra. Vibrating member 21 typically employs piezoelectric ceramics, electromagnets having a vibrating ferromagnetic core, or electric motor 26, such as a small DC motor turning an unbalanced flywheel, and control circuitry 28 for their activation. Vibrating member 21 is energized by battery 30, such as a coin battery. It is housed in a compartment formed below cap 32 attached to the distal face of SB base 22. Operating switch 34, such as a touch switch, is located on top of cap 32. This cap is typically spherical but may take any reasonable shape as long as it includes, or covers the vibrating member. Optionally the operating switch and the battery, or an electrical connector providing for connecting to an external source of electrical power, are installed in a separate housing attachable to a dress of the user. In such a case electrical wires serially connect between the vibrating member and the switch and the battery or the electrical connector. Base 22 and cap 32 are typically made of elastic materials, such as plastic or rubber.

SB 20 is attached to a specific area of a user's skin after the protecting cover that seals the SB base is removed and the active substance is applied to the epidermis. Subsequently, one or more active substance starts diffusing into the skin. Control circuitry 28 is manually activated by the user by means of operating switch 34. The vibrating member then starts vibrating. The mechanical vibrations, although partially dampened by the flexible material from which SB base 22 and covering cap 32 are made of, massage an area covered by SB 20. Optionally, a user may select among a few different rotational rates and several different time cycles to be employed by means of operating switch 34.

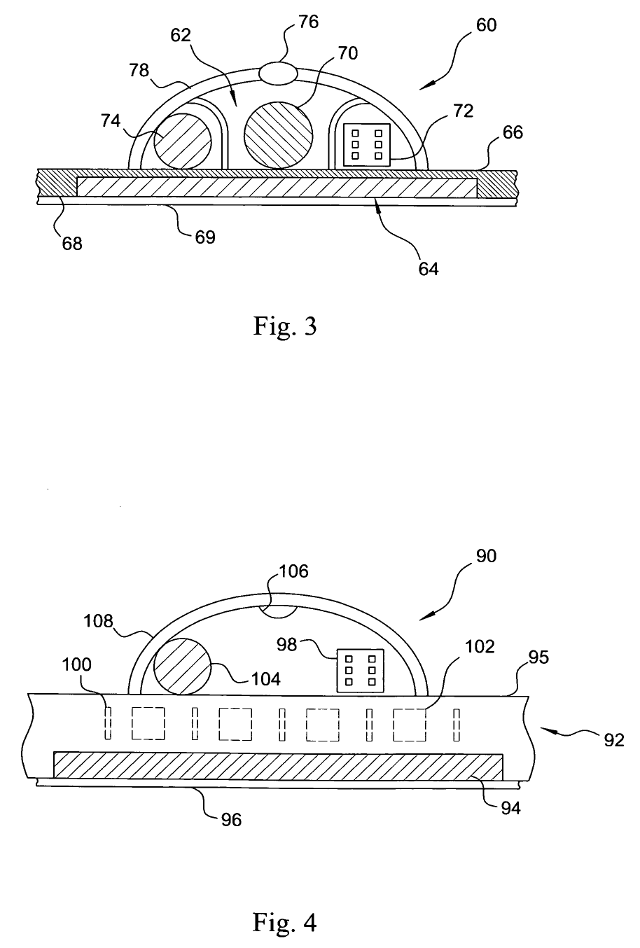

Reference is now made to FIG. 3 showing a sectional view of an SB according to a preferred embodiment of the present invention. SB 60 consists of vibrating member 62 and a layer of active substance contained in depression 64 at the proximal face of SB base 66. The proximal face of rim 68 of SB base 66 is coated with glue. Protecting cover 69 seals the proximal face of SB base 66 in order to protect the active substance. Vibrating member 62 employs electric motor 70, controlled by control circuitry 72 and powered by battery 74. Pressing operating switch 76 located on top of covering cap 78 activates vibrating member 62. The glue coating spans across the proximal face of the rim of SB base 68. Protecting cover 69 is removed before attaching SB 60 to the area to be treated. The adhering coating is made such that SB 60 is firmly attached to the skin of a user after protecting cover 69 is removed. SB 60 is attached to the skin by means of the adhering rim of SB base 66. Removal of protecting cover 69 closes an electrically operated safety circuit, not shown, such as by removing an insulating layer separating between two leads of electric wires. Thus, electric motor 70 is not activated and battery 74 retains its stored energy even if operating switch 76 is accidentally depressed, unless protecting cover 69 has been removed and the SB 60 is firmly attached to the skin of a user. Optionally a semi-permeable sheet or a net, not shown, is disposed between the proximal face of SB base 66 and protecting cover 69 for regulating the efflux of the active material.

Reference is now made to FIG. 4 showing an SB according to another preferred embodiment of the present invention. SB 90 includes vibrating member 92 and a layer of active substance contained in depression 94 located at the proximal face of SB base 95. The proximal face of the rim of SB base 95 is coated with glue and protecting cover 96 is attached to it as is described herein above. Vibrating member 92 consists of two interleaved arrays of electromagnets 100 and 102, embedded in SB base 95. The strengths and polarities of the magnets are controlled by control circuitry 98. The axes of all solenoids of both arrays are parallel to the surface of SB base 95, namely to the surface of the skin area to be treated. All axes of the solenoids of each array are collinear. However the axes of the solenoids of array 100, are perpendicular to the axes of solenoids of array 102. All solenoids of both arrays are independently electrically connected, not shown, to control circuitry 98. Battery 104 energizes vibrating member 92. Removal of protecting cover 96 closes an electrically operated safety circuit, not shown. SB 90 is attached to the skin of a user after protecting cover 96 has been removed. Ingredients of the active substance start diffusing into the dermis layer following such attachment. Pressing operating switch 106 located on top of covering cap 108 activates vibrating member 92. Electric currents the directions and magnitudes of which are controlled by control circuitry 98 are conducted through all solenoids. The electric currents are cyclically conducted in each array in opposing directions, such that magnetic fields of opposing polarities are formed by adjacent solenoids of the same array. The magnitudes and directions of the electrical currents conducted through all solenoids of each array are synchronized such that one or more pairs of adjacent solenoids have equal magnetic intensity but each with an opposing polarity. The magnitudes of the electrical currents of all solenoids need not be the equal. As a result, two-dimensional vibrations are effected within elastic SB base 95 following such activation. Optionally, an alternating current having one or more frequencies and a few different phases is conducted through different solenoids of each array. As SB base 95 is firmly attached to the skin of a user massage of the skin and the underlying muscles below and surrounding SB 90 is effected by these vibrations.

In some embodiments, mixed arrays of fixed magnets and solenoids are applied. In other embodiments the mechanical massage is applied by an electric motor jointly with a magnetic massage. In such embodiments the magnetic polarities are usually normal to the surface of the SB base. Solenoids whose axes are normal to the SB base are employed. Optionally, an electrical heating element is further incorporated providing for warming of the area beneath the SB. In some embodiments the SB is powered by an external power supply or by an input/output port of a computer such as a USB. In such cases a connector is provided on top of a wall of the covering cap or the separate housing for electrical connection to the power supply or to the input/output port. Programming of the control circuitry of the vibrating member is provided in the event that a computer powers the SB. In such cases various vibrating profiles can be selected by the user.

1. A sticky bandage (SB) attachable to a skin of a user, said SB comprises a base having a proximal face and a distal face, wherein a depression accommodated to enclose at least one active substance is located at said proximal face, and wherein a rim of said proximal face is sticky, and wherein a vibrating member disposed in association with said distal face, said SB comprising an electrically operated safety circuit.

2. An SB as in claim 1, wherein said base comprises a first layer and a second layer which is replaceable, and wherein said second layer proximally disposed to said first layer.

3. An SB as in claim 2, wherein said second layer detachably attached to said first layer.

4. An SB as in claim 1, wherein said vibrating member comprises at least two arrays of solenoids embedded in said base.

5. An SB as in claim 1, wherein at least one fixed magnet is embedded in said base.

6. An SB as in claim 1, wherein said vibrating member comprises any item selected from a group of items consisting of a piezoelectric device, a ceramic vibrating element, an electromagnet, a fixed magnet, an electric motor and any combination thereof.

7. An SB as in claim 1, wherein a protecting cover attached to said proximal face.

8. An SB as in claim 7, wherein removing said protecting cover closes said electrically operated safety circuit.

9. An SB as in claim 7, wherein an item selected from a group of items consisting of a semi-permeable sheet and a net, is disposed between said cover and said proximal face.

10. An SB as in claim 1, further comprising an electrical heating element for transferring heat to said skin.

11. An SB as in claim 1, linked to a computer for programming said vibrating member.
